(19) United States
(12) Patent Application Publication
(10) Pub. No.: US 2026/0125350 A1
(43) Pub. Date: May 7, 2026

(54) CENTROMERE PROTEIN CENP-M SMALL MOLECULE INHIBITOR CENPEMLIN, AND PREPARATION METHOD THEREFOR AND USE THEREOF

(71) Applicant: UNIVERSITY OF SCIENCE AND TECHNOLOGY OF CHINA, Hefei (CN)

(72) Inventors: Xuebiao YAO, Hefei (CN); Xing LIU, Hefei (CN); Fengrui YANG, Hefei (CN); Mingming MA, Hefei (CN)

(73) Assignee: UNIVERSITY OF SCIENCE AND TECHNOLOGY OF CHINA, Hefei (CN)

(21) Appl. No.: 19/486,048

(22) PCT Filed: Jul. 27, 2022
(86) PCT No.: PCT/CN2022/108196

§ 371 Date: Nov. 20, 2025

(30) Foreign Application Priority Data
Jul. 11, 2022 (CN) 202210809550.2

(51) Int. Cl. C07D 251/70; A61K 31/5377; A61P 35/00

(52) U.S. Cl. CPC C07D 251/70; A61K 31/5377; A61P 35/00

(57) ABSTRACT

Provided are a centromere protein CENP-M small molecule inhibitor cenpemlin, and a preparation method therefor and the use thereof. A small molecule compound cenpemlin is obtained by means of analyzing the protein plane information of the fine structures of CENP-M and CENP-L and performing targeted screening. The results of biochemical and cytological experiments all indicate that cenpemlin inhibits the proliferation of cancer cells by means of interfering with the interaction between CENP-M and CENP-L, thereby providing a new compound for analyzing and interfering with the rapid proliferation of cancer cells.

### CROSS REFERENCE TO THE RELATED APPLICATIONS

This application is the national phase entry of International Application No. PCT/CN2022/108196, filed on Jul. 27, 2022, which is based upon and claims priority to Chinese Patent Application No. 202210809550.2, filed on Jul. 11, 2022, the entire contents of which are incorporated herein by reference.

### TECHNICAL FIELD

The present invention relates to the field of medicine, and specifically relates to cenpemlin, a small molecule inhibitor of CENP-M, a key centromere protein of mitosis, and a preparation method and use thereof.

### BACKGROUND

Precise cell mitosis ensures the stability of the genome and the maintenance of cell homeostasis. Mitosis produces two genetically identical daughter cells through the equal separation of sister chromatids. The molecular mechanism driving chromosome separation is relatively conserved during the evolution of eukaryotes. The kinetochore is an essential element of this molecular mechanism and can be regarded as a layered structure, with its inner and outer layers directly contacting the centromere chromatin and spindle microtubules, respectively. The inner layer of the kinetochore is composed of at least 16 centromere proteins, which assemble into multi-complexes and directly interact with the centromere chromatin. These complexes include: CENP-H, I, K, M complex, CENP-O, P, Q, U, R complex, CENP-T, W, S, X complex, CENP-L, N complex and CENP-C protein. After these complexes are assembled, they further recruit the remaining kinetochore proteins. Although the interactions between centromere proteins have been widely studied, the specific biological functions of some of these proteins still need to be further explored.

Recently, our group used cryo-electron microscopy to analyze the structure of the CCAN complex. The CCAN complex is in the shape of Chinese character “human”, with CENP-L/N dimers forming an arch, and DNA passing through the bottom of the arch. The CENP-M protein interacts with the other three centromere proteins CENP-H, I, and K to form a complex and become part of the inner layer of the kinetochore. CENP-M binds near the CENP-L/N dimer at the top of the arch, and has an interaction interface with both CENP-L and CENP-N in space. At the same time, CENP-M is localized at the kinetochore throughout the cell cycle, and its function may be constitutive, but the specific molecular mechanism has not yet been fully resolved.

CENP-M was originally named PANE1 (proliferation associated nuclear element 1) and is only conserved in metazoans. Subsequent studies have shown that CENP-M is closely related to CENP-A, CENP-L, CENP-N, and CENP-T. The loss of CENP-M leads to the mislocalization of other CCAN proteins, indicating that CENP-M is essential for the assembly and stability of the inner kinetochore. Although CENP-M is structurally and evolutionarily related to GTPase, but it is not a true enzyme. By forming a quaternary complex with the evolutionarily conserved CENP-H, I, and K proteins, it plays a vital role in a series of important biological processes such as recruiting the chromatin remodeling FACT protein complex, promoting CENP-A nucleosome remodeling, and recruiting spindle checkpoint-related proteins.

There are currently no reports on CENP-M inhibitors.

### SUMMARY

The purpose of the present invention is to provide a compound, an isomer thereof, and a pharmaceutically acceptable salt thereof, and a preparation method and use thereof.

The compound provided by the present invention has a structural formula as shown in Formula I:

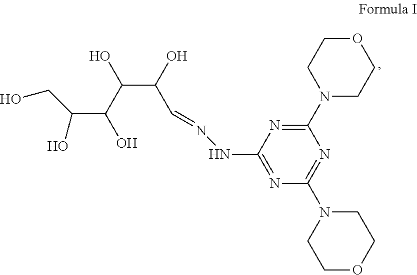 [SMILES: OCC(O)C(O)C(O)C(O)/C=N/Nc1nc(N2CCOCC2)nc(N2CCOCC2)n1]

The compound shown in the above Formula I specifically targets the key centromere protein CENP-M in mitosis, and is a CENP-M inhibitor designated as cenpemlin.

CENP-M is an important member of the inner kinetochore CCAN complex. It was named PANE1 because it was identified in rapidly proliferating cells. Subsequent studies found that CENP-M is a pseudo-GTPase. In recent years, studies on the inner kinetochore CCAN complex have emerged in an endless stream, but its fine structure has not been fully resolved. We use structural biology methods based on cryo-electron microscopy to answer scientific questions. The CCAN complex proteins were expressed and purified using an insect system, and then recombined in vitro to obtain a complete CCAN complex. The three-dimensional image of the complex was collected by cryo-electron microscopy, and then three-dimensional reconstruction was performed. Finally, we obtained the structural information of the CCAN complex containing the CENP-M protein. Based on the fine structure of the interaction interface between CENP-M and CENP-L proteins, we identified a specific CENP-M targeting small molecule compound, cenpemlin, by combining molecular docking analysis, virtual screening, cell phenotype analysis and protein interaction experiments.

The compound shown in Formula I was prepared by a method including the following steps according to the synthetic route shown in FIG. 1:
1) reacting cyanuric chloride and morpholine under alkaline conditions to generate a cyanuric chloride intermediate 1 containing a di-1,4-oxo-nitrogen heterocycle;

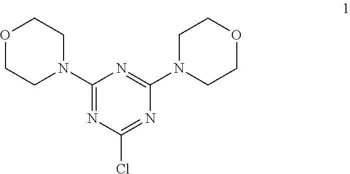 [SMILES: Clc1nc(N2CCOCC2)nc(N2CCOCC2)n1]

2) subjecting the cyanuric chloride intermediate 1 containing a di-1,4-oxo-nitrogen heterocycle to a substitution reaction with hydrazine hydrate to generate 1,3(1,4-oxo-nitrogen heterocycle)-5-hydrazine polycyanuric acid (compound 2);

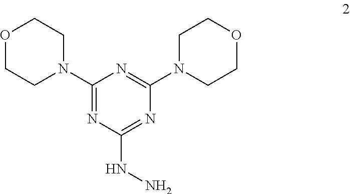 [SMILES: NNc1nc(N2CCOCC2)nc(N2CCOCC2)n1]

3) subjecting 1,3(1,4-oxo-nitrogen heterocycle)-5-hydrazine polycyanuric acid to a condensation reaction with a hexagonal monosaccharide in glacial acetic acid to obtain the compound shown in Formula I.

In step 1) of the above method, the molar ratio of cyanuric chloride to morpholine can be 1:2-2.5, specifically 1:2; The reaction is carried out in an ice-water bath, and the reaction time can be 1-1.5 h, specifically 1 h;

The alkaline condition is provided by sodium bicarbonate; the molar ratio of sodium bicarbonate to morpholine can be 1:1.0-1.2, specifically 1:1;

In step 2) of the above method, the molar ratio of the cyanuric chloride intermediate 1 containing a di-1,4-oxo-nitrogen heterocycle to hydrazine hydrate is 1:1-2;

The reaction temperature is 40° C.-60° C., the time is 3-4 h, specifically 3 h;

In step 3) of the above method, the ratio of 1,3(1,4-oxazaheterocyclic)-5-hydrazine cyanohydride to hexacarbon monosaccharide can be 10 mmol: 1.8-2.0 g, specifically 10 mmol:1.8 g; The hexacarbon monosaccharide that can be used as raw material includes but is not limited to D-glucose, D-galactose, and D-mannose; The reaction temperature is 40° C.-60° C., the time is 4-5 h, specifically 4 h.

The use of the compound shown in formula I, isomers thereof and pharmaceutically acceptable salts thereof in the preparation of products with the following functions also belongs to the protection scope of the present invention:
1) centromere protein CENP-M inhibitor;
2) reagents for inhibiting cell mitosis;
3) products for inhibiting tumor cell proliferation;
4) products for preventing and/or treating cancer;
In 2), the inhibition of cell mitosis is achieved by interfering with CENP-M-mediated kinetochore assembly, specifically inhibiting the interaction between CENP-M and CENP-L;
In 3), the tumor cells may be epithelial cancer cells; specifically cervical cancer cells, liver cancer cells, breast cancer cells, etc.;
4) The cancer may be epithelial cancer, specifically cervical cancer, liver cancer, breast cancer, etc.

The present invention also provides a reagent for inhibiting cell mitosis, the reagent contains the compound shown in formula I, isomers thereof or pharmaceutically acceptable salts thereof.

The present invention also provides a product for inhibiting tumor cell proliferation, the product for inhibiting tumor cell proliferation contains the compound shown in formula I, isomers thereof or pharmaceutically acceptable salts thereof.

The present invention also provides a product for preventing and/or treating cancer, which contains a compound shown in Formula I, an isomer thereof, or a pharmaceutically acceptable salt thereof.

The present invention also provides a method for inhibiting the interaction between CENP-M and CENP-L, including: adding the compound of formula I of claim 1, isomers thereof or a pharmaceutically acceptable salt thereof to a system containing CENP-M and CENP-L, and incubating the system.

The CENP-M inhibitory organic small molecule compound provided by the present invention, after being added to the culture medium and binding to the CENP-M protein, causes the chromosomes misalignment and further causes the cells to have a phenotype of delayed mitosis and lagging chromosomes.

Real-time live cell imaging found that cells treated with cenpemlin would produce chromosome misalignment and spindle instability, and also lead to a phenotype of delayed mitosis. These phenotypes of induced abnormal mitosis in cells are consistent with the phenotype of CENP-M knockdown.

Cenpemlin can inhibits the interaction between CENP-M and CENP-L when treated at a concentration of 1 μM. Using the effective inhibitory effect of cenpemlin on CENP-M/CENP-L, the function of CENP-M in kinetochore assembly and chromosome arrangement can be further studied.

The CENP-M small molecule inhibitor cenpemlin of the present invention will play an important role in cell biology research, and its efficacy in regulating tumor cell proliferation can lay the foundation for the development of new chemotherapy drugs.

The present invention analyzes the protein surface information of the fine structure of CENP-M and CENP-L and screens the small molecule compound cenpemlin in a targeted manner. The results of biochemical and cytological experiments show that cenpemlin inhibits the proliferation of cancer cells by intervening in the interaction between CENP-M and CENP-L, providing a new compound for analyzing and intervening in the rapid proliferation of cancer cells.

### BRIEF DESCRIPTION OF THE DRAWINGS

FIG. 1 is a synthetic route of the CENP-M inhibitor shown in Formula 1 of the present invention.

FIG. 2 is the nuclear magnetic resonance identification result of the small molecule compound cenpemlin.

FIG. 3 is the phenotypic analysis of the small molecule compound cenpemlin on cell mitosis.

FIG. 4 is the real-time imaging analysis of the small molecule compound cenpemlin blocking cell mitosis.

FIG. 5 is the elution effect analysis of the small molecule compound cenpemlin.

FIG. 6 shows that the small molecule compound cenpemlin inhibits the interaction between CENP-M and CENP-L.

FIG. 7 is a statistical analysis of the small molecule compound cenpemlin inhibiting the proliferation of cancer cells.

FIGS. 8A-8B show an experiment of the small molecule compound cenpemlin inhibiting the proliferation of liver cancer cells in mice.

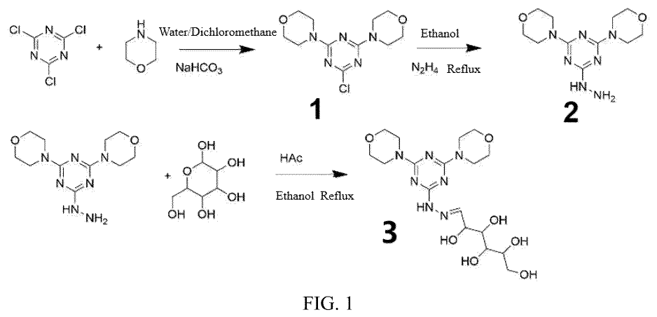

### DETAILED DESCRIPTION OF THE EMBODIMENTS

The experimental methods in the following examples, unless otherwise specified, are conventional methods.

The present invention is further described below with reference to the examples, but the present invention is not limited in any way. Any changes or improvements made based on the teachings of the present invention fall within the scope of protection of the present invention.

The accession number of CENP-M used in the following examples in the NCBI database is Accession: NP_076958.1 (Update: 27 Jun. 2022)

The accession number of CENP-L used in the following examples in the NCBI database is Accession: NP_001164653.1 (update: 30 Jan. 2022)

### Example 1: Synthesis of the Small Molecule Compound Cenpemlin

Dissolve cyanuric chloride (10.0 g, 0.054 mol) in 150 mL of dichloromethane and cool the solution to below 5° C. in an ice-water bath. Dissolve morpholine (9.50 mL, 0.108 mol) and sodium bicarbonate (9.1 g, 0.108 mol) in 100 mL of water and cool the solution to below 5° C. in an ice-water bath. Add the aqueous solution dropwise to the dichloromethane solution in an ice-water bath with rapid stirring and continue stirring for 1 h. Separate the two phases and wash the organic phase with 50 mL of water twice. Concentrate the organic phase to 20 mL by rotary evaporation, add 50 mL of petroleum ether to the organic phase, filter the resulting white precipitate and wash it with cold water and cold petroleum ether, and dry it to obtain 14.7 g of white solid, i.e., intermediate 1, with a yield of 95%. Nuclear magnetic hydrogen spectrum: 1H NMR (400 MHz, CDCl₃) δ 3.80-3.68 (m, 16H).

Add 25 mL of ethanol to the intermediate product 1 (5.7 g, 20 mmol), add 5 mL of hydrazine hydrate, heat the solution to 40° C.-60° C. and reflux for 3 hours, then cool to room temperature, and a white solid gradually precipitates. Filter and wash with 20 mL of ethanol, wash twice, and dry to obtain 5.2 g of a white solid product, namely compound 2, with a yield of 93%. Nuclear magnetic hydrogen spectrum: 1H NMR (400 MHz, CDCl₃) δ 3.80-3.68 (m, 16H), 4.77 (d, 2H), 9.39 (t, 1H).

Compound 2 (2.8 g, 10 mmol) was added to 25 mL of ethanol, 10 mmol of hexose (1.8 g) was added, and 0.2 mL of glacial acetic acid was added. The solution was heated to 40° C.-60° C. and refluxed for 4 hours, then cooled to room temperature, and a white solid gradually precipitated. Filter and wash with 20 mL of ethanol, wash twice, and obtain a white solid product. The product can be purified by recrystallization with ethanol/ethyl acetate. Depending on the type of hexose used, the yield of the obtained product is between 70-85%. Hexose monosaccharides that can be used as raw materials include but are not limited to D-glucose, D-galactose, and D-mannose.

The small molecule structure was identified using a Bruker AV-500 NMR spectrometer. According to the chemical shift of the hydrogen spectrum and the integrated area of the peak, it was found that the structure of the small molecule conformed to the structure of formula I.

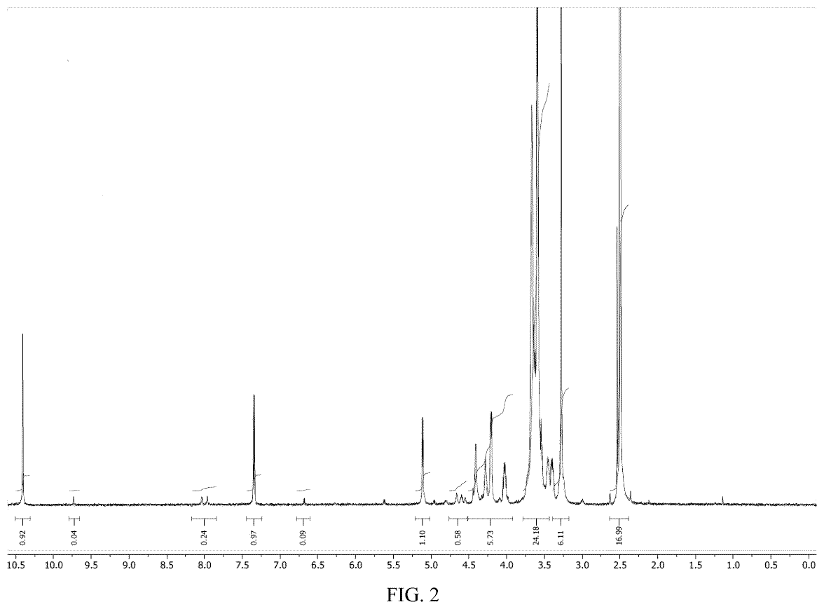

FIG. 2 shows the NMR identification results of the small molecule compound cenpemlin.

The structural formula of the compound and the assignment of its 600 MHz¹ NMR spectrum are as follows:

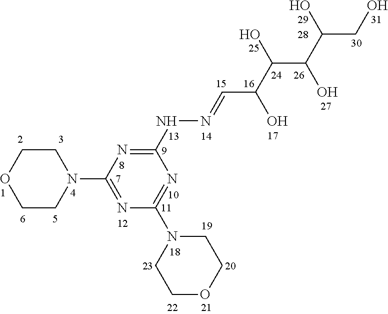 [SMILES: OCC(O)C(O)C(O)C(O)/C=N/Nc1nc(N2CCOCC2)nc(N2CCOCC2)n1]

The attribution of its 600 MHz¹ spectrum is as follows:

XH NMIR (600 MHz¹, DMSO-d6) δ 3.01 (26, m, 1CH), δ 3.59 (3, 5, 19, 23, m, 4cH2), δ 3.67 (3, 5, 19, 23, m, 4cH2), δ 4.34 (30, t, 1CH2), δ 4.43 (28, m, 1CH), δ 4.62 (26, m, 1CH), δ 4.87 (16, d, 1CH), δ 6.25 (15, d, 1CH), δ 8.05 (13, s. 1NH)

### Example 2. The Small Molecule Compound Cenpemlin Causes Chromosome Misalignment

### 1. Experimental Steps

1) HeLa cells stably expressing GFP-CENP-N were seeded on a round coverslip with a diameter of 12 mm;
2) after 24 h, Thymidine was added to the cells at a final concentration of 2 mM for 14-16 h;
3) the cells were washed three times with PBS prewarmed at 37° C. to elute Thymidine, and fresh culture medium was added to continue culturing for 8 h;
4) cempelin was added to the experimental group at a final concentration of 1 μM, and the control group was added with an equal volume of DMSO for 1 h;
5) the cells were fixed with PBS buffer containing 3.7% formaldehyde for 10 min;
6) after 0.2% TritonX-100 permeabilization and 1% BSA blocking, ACA antibody was incubated at room temperature for 1 h;
7) DAPI was stained for 3 min and then the slides were sealed;
8) the slides were placed on the DV microscope stage and imaged by a 60×, NA=1.42 lens.

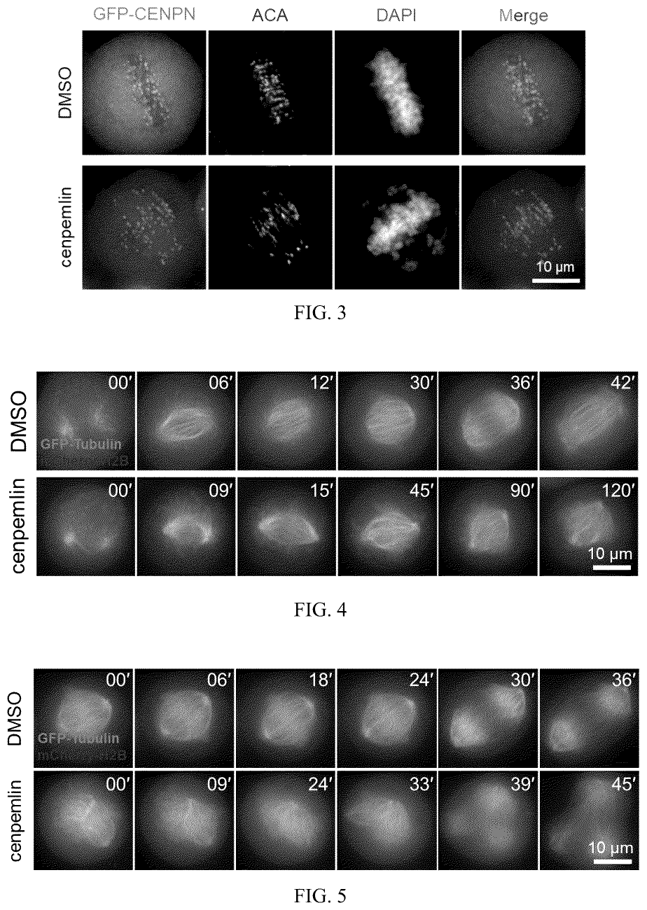

### 2. The Results are Shown in FIG. 3.

After HeLa cells were treated with cenpemlin, a large number of chromosomes that were not arranged at the equatorial plate were observed. Using GFP-CENP-N as a representation of the CENP-L/N complex, kinetochores that were not arranged in the equatorial plate region were observed, indicating that cenpemlin treatment of cells would lead to abnormal chromosome alignment in cell mitosis.

### Example 3. The Small Molecule Compound Cenpemlin Causes Mitotic Arrest

### 1. Experimental Steps

1) Inoculate HeLa cells into 35 mm live cell culture dishes;
2) transfect plasmid DNA: GFP-tubulin and mCherry-H2B after 24 h (the density of transfected plasmid cells is 70%-80%);
3) replace with fresh culture medium 6 h after transfection and add 2 mM Thymidine to treat cells for 14-16 h;
4) wash cells 3 times with PBS preheated at 37° C. to release cells, then replace with fresh culture medium to continue culturing;
5) turn on the Applied Precision Personal DV microscope and thermostat to stabilize the temperature at 37° C.;
6) 8-9 h after release, replace the cell culture medium with CO₂-independent Medium, add cenpemlin with a final concentration of 1 μM or the same volume of DMSO;
7) place the cells on the 37° C. thermostat stage of the DV microscope and shoot real-time under a 60×, NA=1.42 lens, taking one frame every 3 min.

### 2. The Results are Shown in FIG. 4.

From the perspective of chromosome movement, cenpemlin treatment caused some chromosomes in the cells to be unable to line up correctly, and also caused obvious mitotic delays. The cells did not enter the late stage 120 minutes after the nuclear membrane ruptured.

This conclusion also confirmed that some previous studies believed that CENP-M has a certain function in the spindle assembly checkpoint.

### Example 4. Detection of Elution Effect of Small Molecule Compound Cenpemlin

### 1. Experimental Steps

1) Seed HeLa cells into 35 mm live-cell imaging dishes;
2) transfect plasmid DNA after 24 h: GFP-tubulin and mCherry-H2B (the density of transfected plasmid cells is 70%-80%);
3) replace with fresh culture medium 6 h after transfection and add MG132 at a final concentration of 20 μM and cenpemlin or DMSO to treat the cells for 1 h;
4) turn on the Applied Precision Personal DV microscope and the thermostat to stabilize the temperature at 37° C.;
5) wash the cells three times with PBS prewarmed at 37° C. to release the cells, and then replace with fresh culture medium to continue culturing;
6) immediately after release, replace the cell culture medium with CO₂-independent Medium;
7) place the cells on the 37° C. thermostat stage of the DV microscope and imaging in real time by a 60×, NA=1.42 lens, taking one frame every 3 min.

### 2. The Results are Shown in FIG. 5.

Cenpemlin-treated cells exit mitosis after 45 min, but the cells exhibit a multicellular phenotype, that is, the genome is unevenly distributed, because cenpemlin-treated cells exhibit multipolar spindles.

### Example 5. Small Molecule Compound Cenpemlin Affects the Interaction Between CENP-M and CENP-L

### 1. Experimental Steps

1) Inoculate HEK293T cells into 6 cm culture dishes;
2) transfect plasmid DNA: GFP-CENP-M and FLAG-CENP-L after 24 h (the density of cells is 70%-80%);
3) replace with fresh culture medium 4 h after transfection and continue to culture for 24 h;
4) add cenpemlin with a final concentration of 1 μM or the same volume of DMSO to treat cells for 1 h;
5) scrape the cells with a cell scraper, collect them in a centrifuge tube, and remove the culture medium by centrifugation at 1,000 rpm;
6) add cell lysis buffer for ultrasonic disruption, and centrifuge at 12,000 rpm for 10 min;
7) take the supernatant and incubate with FLAG beads for 4 h;
8) wash the beads for three times with cell lysis buffer, boil the sample with sample buffer, and then perform electrophoresis and immunoblot analysis.

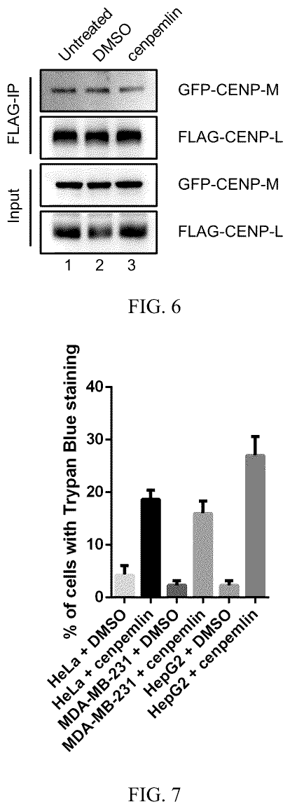

### 2. The Results are Shown in FIG. 6.

After cenpemlin treatment (lane 3), the interaction between CENP-M and CENP-L was weakened compared with the control group (lane 2), indicating that 1 μM cenpemlin is sufficient to disrupt the interaction between CENP-M and CENP-L. This result is consistent with the cell phenotype experiment as previously mentioned, indicating that cenpemlin inhibits tumor cell proliferation by interfering with the interaction between CENP-M and CENP-L.

### Example 6. Broad-Spectrum Anti-Cancer Effect of the Small Molecule Cenpemlin

### 1. Experimental Steps

1) HeLa cells (cervical cancer cells), MDA-MB-231 cells (triple negative breast cancer cells) and HepG2 cells (liver cancer cells) were seeded into 6 cm culture dishes;
2) The small molecule compound cenpemlin or DMSO was added at a final concentration of 1 μM to treat the cells for 24 h;
3) The cells were digested with trypsin and the cell suspension was taken for trypan blue staining;
4) The blue-stained cells were observed and counted under an ordinary optical microscope.

### 2. The Results are Shown in FIG. 7.

After cenpemlin treatment, HeLa cells, MDA-MB-231 cells and HepG2 cells all showed different proportions of trypan blue staining increase. This result is consistent with the cell phenotype experiment as previously mentioned, indicating that cenpemlin has a broad-spectrum inhibitory effect on cancer cell proliferation.

### Example 8. The Small Molecule Compound Cenpemlin Inhibits the Proliferation of Liver Cancer Cells

### 1. Experimental Steps

1) According to the published experimental protocol (Chen et al., 2011. Cancer Res), we implanted luciferase-stable liver cancer cells MHCC97-H cells (5×10⁶ in 0.1 mL saline) into the liver of 6-week-old female NOD/SCID mice. The experiment was divided into three groups, namely the DMSO control group, the paclitaxel group and the cenpemlin group (2 mg/kg), with 10 mice in each group.
2) After 14 days of modeling (successful modeling of luciferase test), the DMSO control group, the paclitaxel group and the cenpemlin experimental group (2 mg/kg) were intraperitoneally injected (0.2 mL per mouse; 0.1 mL was injected on both sides) once a day for three days.
3) After four injections every other day (the 11th day of administration), the luciferase test was performed.

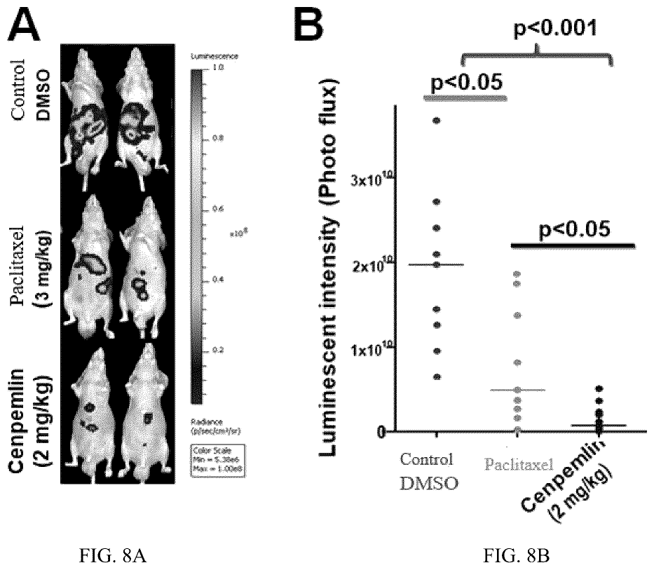

### 2. The Results are Shown in FIGS. 8A-8B.

FIG. 8A is the luciferase imaging image on the 11th day after administration. FIG. 8B is the statistical analysis graph. The results showed that the number of cancer cells in the liver of mice in the paclitaxel and small molecule compound cenpemlin groups was significantly reduced. The above results show that the small molecule compound cenpemlin can effectively inhibit the proliferation of liver cancer cells in mice, providing a new perspective and target for the screening and development of anticancer drugs.

### INDUSTRIAL APPLICATION

The present invention identified the small molecule compound cenpemlin through targeted screening by using protein surface information from the fine structure of CENP-M and CENP-L. Biochemical and cytological experiments demonstrated that cenpemlin inhibits cancer cell proliferation by interfering with the interaction between CENP-M and CENP-L, providing a novel compound for analyzing and intervening in the rapid proliferation of cancer cells.

What is claimed is:

1. A compound of formula I, isomers thereof, and pharmaceutically acceptable salts thereof:

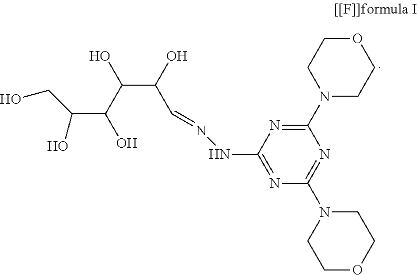 [SMILES: OCC(O)C(O)C(O)C(O)/C=N/Nc1nc(N2CCOCC2)nc(N2CCOCC2)n1]

2. A method for preparing the compound of formula I according to claim 1, comprising the following steps:
1) reacting cyanuric chloride and morpholine under an alkaline condition to generate a cyanuric chloride intermediate 1 containing a di-1,4-oxo-nitrogen heterocycle; 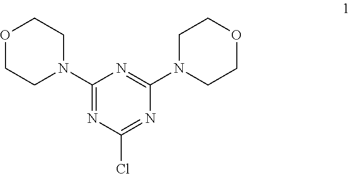 [SMILES: Clc1nc(N2CCOCC2)nc(N2CCOCC2)n1]
2) subjecting the cyanuric chloride intermediate 1 containing the di-1,4-oxo-nitrogen heterocycle to a substitution reaction with hydrazine hydrate to generate compound 2, wherein the compound 2 is 1,3(1,4-oxo-nitrogen heterocycle)-5-hydrazine cyanuric acid; 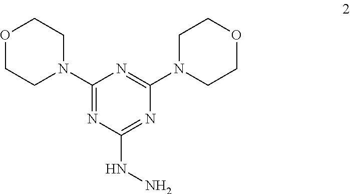 [SMILES: NNc1nc(N2CCOCC2)nc(N2CCOCC2)n1]
3) subjecting the 1,3(1,4-oxo-nitrogen heterocycle)-5-hydrazine cyanuric acid to a condensation reaction with a hexacarbon monosaccharide in glacial acetic acid to obtain the compound of formula I.

3. The method according to claim 2, wherein in the step 1), a molar ratio of the cyanuric chloride to the morpholine is 1: 2-2.5;
the reaction is carried out under an action of an ice-water bath, and a reaction time is 1-1.5 h;
the alkaline condition is provided by sodium bicarbonate.

4. The method according to claim 2, wherein in the step 2), a molar ratio of the cyanuric chloride intermediate 1 containing the di-1,4-oxo-nitrogen heterocycle to the hydrazine hydrate is 1: 1-2;
a reaction temperature is 40° C.-60° C., a reaction time is 3-4 h.

5. The method according to claim 2, wherein in the step 3), a ratio of the 1,3(1,4-oxo-nitrogen heterocycle)-5-hydrazine cyanuric acid to the hexacarbon monosaccharide is 10 mmol: 1.8-2.0 g;
a reaction temperature is 40° C.-60° C., a reaction time is 4-5 h.

6. A method for preparing a product, comprising using the compound of formula I according to claim 1, the isomers thereof, and the pharmaceutically acceptable salts thereof, wherein the product comprises:
1) an inhibitor of centromere protein CENP-M;
2) an agent for inhibiting cell mitosis;
3) a product for inhibiting tumor cell proliferation; and
4) a product for preventing and/or treating a cancer.

7. The method according to claim 6, wherein inhibiting cell mitosis is achieved by interfering with CENP-M-mediated kinetochore assembly.

8. The method according to claim 6, wherein tumor cells are epithelial cancer cells, and the epithelial cancer cells comprise cervical cancer cells, liver cancer cells, and breast cancer cells.

9. The method according to claim 9, which wherein the cancer is epithelial cancer, and the epithelial cancer comprises cervical cancer, liver cancer, and breast cancer.

10. A reagent for inhibiting cell mitosis, comprising the compound of formula I according to claim 1, the isomers thereof, and the pharmaceutically acceptable salts thereof.

11. A product for inhibiting tumor cell proliferation, comprising the compound of formula I according to claim 1, the isomers thereof, and the pharmaceutically acceptable salts thereof.

12. A product for preventing and/or treating cancer, comprising the compound of formula I according to claim 1, the isomers thereof, and the pharmaceutically acceptable salts thereof.

13. A method for inhibiting an interaction between CENP-M and CENP-L, comprising: adding the compound of formula I according to claim 1, the isomers thereof, and the pharmaceutically acceptable salts thereof to a system containing the CENP-M and the CENP-L, and incubating the system.

14. The method according to claim 3, wherein in the step 2), a molar ratio of the cyanuric chloride intermediate 1 containing the di-1,4-oxo-nitrogen heterocycle to the hydrazine hydrate is 1: 1-2;
a reaction temperature is 40° C.-60° C., a reaction time is 3-4 h.

15. The method according to claim 3, wherein in the step 3), a ratio of the 1,3(1,4-oxo-nitrogen heterocycle)-5-hydrazine cyanuric acid to the hexacarbon monosaccharide is 10 mmol: 1.8-2.0 g;
a reaction temperature is 40° C.-60° C., a reaction time is 4-5 h.
